(19) United States
(12) Patent Application Publication
(10) Pub. No.: US 2018/0036534 A1
(43) Pub. Date: Feb. 8, 2018

(54) WEARABLE DEVICE HAVING LOW-FREQUENCY GENERATION FUNCTION, AND HEALTH CARE SYSTEM USING THE SAME

(71) Applicant: PIOMED CO., LTD., Seoul (KR)

(72) Inventor: Seung-Chul SHIN, Seoul (KR)

(21) Appl. No.: 15/557,186

(22) PCT Filed: Feb. 24, 2016
(86) PCT No.: PCT/KR2016/001812

§ 371 Date: Sep. 11, 2017

(30) Foreign Application Priority Data
Mar. 11, 2015 (KR) 10-2015-0033870

(51) Int. Cl. A61N 1/36; A61B 5/00; A61B 5/024; A61N 1/04

(52) U.S. Cl. CPC A61N 1/36014; A61N 1/0456; A61B 5/681; A61B 5/02438; A61B 5/0022

(57) ABSTRACT

The present invention relates to a wearable device having a low frequency generation function and a health care system using the same, and more particularly, to a wearable device having a low frequency generation function and a health care system using the same capable of effectively preventing nausea and vomiting due to motion sickness, morning sickness, drugs, or the like by transmitting a low frequency to the median nerve of the wrist.

### TECHNICAL FIELD

The present invention relates to a wearable device having a low frequency generation function and a health care system using the same, and more particularly, to a wearable device having a low frequency generation function and a health care system using the same capable of effectively preventing nausea and vomiting due to motion sickness, morning sickness, drugs, or the like by transmitting a low frequency to the median nerve of the wrist.

### BACKGROUND ART

In general, morning sickness experienced by pregnant women usually appears in the early stage of pregnancy, and the cause thereof is known as an increase in a placenta-secreted hormone (HCG) or mental anxiety or stress due to pregnancy. The morning sickness usually appears as syndromes such as vomiting, sickness, nausea, anorexia, and a change in the taste of food, but normally, most of the symptoms disappear within 5 months.

According to a survey of the morning sickness experience of the pregnant women, the experience or relief of morning sickness varies according to age, first or easy childbirth, the presence of occupation, education levels, income levels, and residences of the pregnant women, and the experienced symptoms also vary according to the stress or family composition due to pregnancy and gender of the birth.

Due to the morning sickness, the pregnant women become more susceptible to smell, and thus, preference for certain foods is decreased or appetite disappears, and as a result, food intake is irregular and a body weight is reduced. Further, when vomiting is worse, the body may lose moisture or minerals and become dehydrated in some cases. Since this may have negative effects on the development and nutritional status of the fetus as well as a fear of pregnancy or other mental conflicts, clinical and nutritional management need to be performed when morning sickness becomes severe.

The solution for alleviating symptoms of morning sickness has mainly used oriental medicine, medicines, or intravenous injection. Vitamin B6 is administered to alleviate symptoms of pregnant women with severe vomiting and nausea. However, drug abuse, including these supplements, may cause undesirable results for the fetus, like drinking or smoking, and drug prescriptions need to be used restrictively under the supervision of a physician.

Also, referring to Korean Patent Laid-Open Publication No. 2001-0032517, although not an apparatus for treating morning sickness, there is disclosed a treatment apparatus for treating vomiting and nausea by applying a low frequency current to a body. Such a conventional treatment apparatus is in the form of one apparatus and includes an oscillator for oscillating a low frequency and a treatment electrode connected to the oscillator and transmitting the low frequency oscillated from the oscillator to the body. However, in such a conventional treatment apparatus, since the oscillator and the treatment electrode are individually provided, the oscillator and the treatment electrode are generally installed in a designated place such as a hospital, because the size is large.

However, whenever the pregnant woman shows symptoms of morning sickness such as vomiting or nausea, it is practically impossible to use conventional treatment apparatus by visiting the hospital promptly. As a result, it is difficult to treat the morning sickness using the conventional treatment apparatus. Therefore, it is urgent to develop a therapeutic apparatus that enables the oscillator and the treatment electrode to be integrally portable and can be easily used.

On the other hand, recently, portable wearable devices have been excitedly released based on large companies. Most of these wearable devices measure the user's momentum to show the calories consumed during a day, or measure the user's pulse to provide a motion guide. In addition, such wearable devices measure bio-signals such as a pulse and a body temperature, analyze the bio-signals, and provide a function of notifying health problems to the user.

However, these wearable devices have only a function of simply measuring the bio-signals such as a pulse and a body temperature and notifying the bio-signals to the outside, but have no separate function to alleviate symptoms of morning sickness such as vomiting and nausea. As a result, there has been a continuous need to add a function for alleviating symptoms of morning sickness such as vomiting and nausea to the portable wearable devices.

### DISCLOSURE

### Technical Problem

The present invention is directed to provide a wearable device having a low frequency generation function and a health care system using the same capable of effectively preventing nausea and vomiting due to motion sickness, morning sickness, drugs, or the like by allowing a portable wearable device to transmit a low frequency to the median nerve of the wrist.

Further, the present invention is directed to provide a wearable device having a low frequency generation function and a health care system using the same capable of measuring user's bio-signals to allow a user or a protector to continuously monitor the bio-signals, thereby rapidly notifying a dangerous situation to the outside when the user is in the dangerous situation.

### Technical Solution

One aspect of the present invention provides a wearable device having a low frequency generation function, in which the wearable device includes a main body provided on the wrist and embedded with a low frequency generating unit generating a low frequency; a wear control unit provided in the main body to control the low frequency generating unit to generate the low frequency; a belt member having a first belt connected to one side of the main body to surround one side of the outer periphery of the wrist and a second belt connected to the other side of the main body to surround the other side of the outer periphery of the wrist; a coupling member coupling the first belt and the second belt; and a treatment electrode unit provided to a portion of the coupling member facing the wrist and transmitting the low frequency generated by the low frequency generating unit to the wrist.

The coupling member may include a coupling guide portion coupled to the first belt and having the treatment electrode unit at the portion facing the wrist; a pair of coupling protrusions protruding from both sides of the coupling guide portion in a direction of the outer periphery of the second belt which is positioned to be stacked on the outer periphery of the first belt; and a coupling buckle hinge-coupled between the pair of coupling protrusions and rotating to press or not the outer periphery of the second belt which is positioned to be stacked on the outer periphery of the first belt.

The belt member may further include a sliding contact portion provided in an inner longitudinal direction of the first belt, positioned to be exposed to the outside along the inner periphery of the first belt, and electrically connected to the low frequency generating unit, and the coupling member may further include the coupling guide portion coupled to slide along the inner periphery of the first belt; a pair of hinge coupling portions provided between the coupling guide portion and the coupling protrusion to face the outer periphery of the first belt; and a fixing buckle hinge-coupled between the pair of hinge coupling portions and rotating to press or not the outer periphery of the first belt, and the treatment electrode unit may include an electrode portion positioned at the coupling guide portion and a contact portion protruding from the electrode portion in the sliding contact portion direction by penetrating the coupling guide portion.

A pair of treatment electrode units may be configured and a pair of sliding contact portions may be configured to contact the treatment electrode units, respectively.

The treatment electrode unit may be positioned to face the median nerve of the wrist.

A measuring unit measuring biometric information of the user may be provided inside the main body facing the wrist.

An output unit outputting various kinds of data to the outside may be further included outside the main body positioned at an opposite side to the wrist, and the wear control unit may output the biometric information measured by the measuring unit to the output unit.

The measuring unit may measure a pulse of the wrist or a body temperature of the wrist.

A pedometer may be provided in the main body.

Another aspect of the present invention provides a health care system using the wearable device having a low frequency generation function, in which the health care system includes the wearable device having a low frequency generation function including the wear control unit receiving the biometric information from the measuring unit and a first short-range communication unit receiving health information data including the biometric information from the wear control unit to output the received health information data to the outside through a first wireless communication network; a first terminal including a second short-range communication unit connected wirelessly to the first wireless communication network and receiving the health information data from the first short-range communication unit, a first long-range communication unit outputting the health information data to the outside through a second wireless communication network, and a first control unit controlling the first long-range communication unit to output the health information data received from the second short-range communication unit to the outside; and a second terminal including a second long-range communication unit connected wirelessly to the second wireless communication network and receiving the health information data from the first long-range communication unit, a terminal output unit outputting the health information data received from the second long-range communication unit to the outside, and a second control unit controlling the terminal output unit to output the health information data received from the second long-range communication unit to the outside.

The first wireless communication network may be configured by a wireless communication network using Bluetooth and the second wireless communication network may be configured by a mobile communication network including any one selected from a 2G network, a 3G network, and a 4G network (an LTE network).

Any one control unit selected from the wear control unit, the first control unit, and the second control unit may store a safe range for the biometric information and generate a warning message when the biometric information deviates from the safe range, and the warning message may be included in the health information data.

The terminal output unit may include a speaker outputting the warning message as a sound or a vibration unit outputting the warning message as a vibration, or a display unit outputting the warning message as an image.

### Advantageous Effects

According to the present invention, it is possible to easily transmit a low frequency generated from the low frequency generating unit provided in the wearable device having the low frequency generation function, thereby efficiently preventing nausea and vomiting due to motion sickness, morning sickness, or drugs.

Further, since the coupling member of the wearable device having the low frequency generation function slides along the first belt, the coupling member may be positioned to face the median nerve of the wrist, thereby allowing the treatment electrode unit provided in the coupling member to easily transmit the low frequency to the median nerve of the wrist. Further, the coupling member positioned to face the median nerve of the wrist is configured to be fixed to the first belt by rotating the fixing buckle to press the first belt, thereby firmly fixing the coupling member while being positioned to face the median nerve of the wrist.

Further, in the present invention, the wearable device having the low frequency generation function is configured to measure the biometric signal of the user and notify the biometric signal to the user or the protector as well as transmit the low frequency to the median nerve of the wrist, thereby continuously monitoring the health condition of the user by the user or the protector and rapidly notifying a dangerous situation to the outside when the user is in the dangerous situation.

### DESCRIPTION OF DRAWINGS

FIG. 1 is a diagram schematically illustrating a wearable device having a low frequency generation function according to a preferred embodiment of the present invention.

FIG. 2 is a diagram schematically illustrating a state in which a first belt is coupled to a sliding contact portion of a belt member in the wearable device having the low frequency generation function according to the preferred embodiment of the present invention.

FIG. 3 is a diagram schematically illustrating a state in which a coupling guide portion of a coupling member is slidably coupled to the first belt to the belt member in the wearable device having the low frequency generation function according to the preferred embodiment of the present invention.

FIG. 4 is a diagram for describing a state in which a treatment electrode unit is coupled to the coupling member in the wearable device having the low frequency generation function according to the preferred embodiment of the present invention.

FIG. 5 is a diagram schematically illustrating a state in which the coupling member is fixed to the first belt in the wearable device having the low frequency generation function according to the preferred embodiment of the present invention.

FIG. 6 is a cross-sectional view illustrating a state in which the coupling member is fixed to the first belt in the wearable device having the low frequency generation function according to the preferred embodiment of the present invention.

FIG. 7 is a diagram schematically illustrating a state in which a second belt is stacked on the first belt in the wearable device having the low frequency generation function according to the preferred embodiment of the present invention.

FIG. 8 is a cross-sectional view illustrating a state in which a coupling buckle is fixed to the second belt in the wearable device having the low frequency generation function according to the preferred embodiment of the present invention.

FIG. 9 is a diagram schematically illustrating a health care system using a wearable device having a low frequency generation function according to a preferred embodiment of the present invention.

FIG. 10 is a block diagram schematically illustrating the wearable device having the low frequency generation function of FIG. 9.

FIG. 11 is a diagram schematically illustrating a display unit included in the wearable device having the low frequency generation function of FIG. 9.

FIG. 12 is a block diagram schematically illustrating a first terminal in the health care system using the wearable device having the low frequency generation function according to the preferred embodiment of the present invention.

FIG. 13 is a block diagram schematically illustrating a second terminal in the health care system using the wearable device having the low frequency generation function according to the preferred embodiment of the present invention.

FIG. 14 is a schematic flowchart for describing the health care system using the wearable device having the low frequency generation function according to the preferred embodiment of the present invention.

### MODES OF THE INVENTION

Hereinafter, a wearable device having a low frequency generation function according to a preferred embodiment of the present invention will be described in more detail with reference to the accompanying drawings.

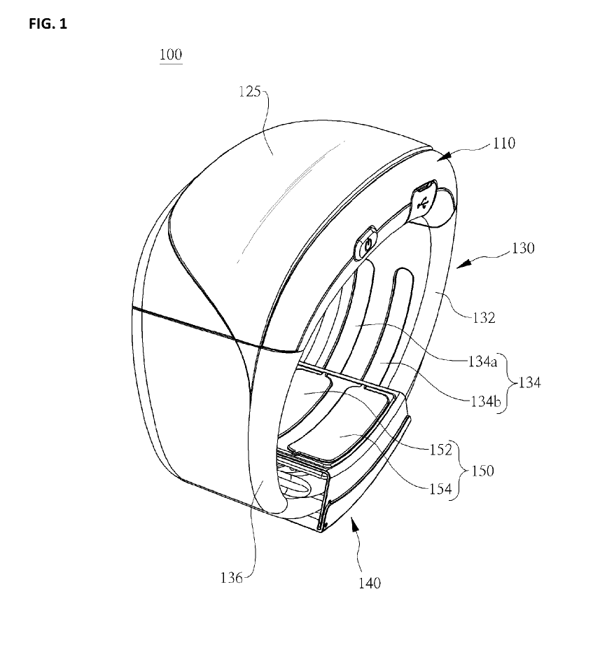

FIG. 1 is a diagram schematically illustrating a wearable device having a low frequency generation function according to a preferred embodiment of the present invention.

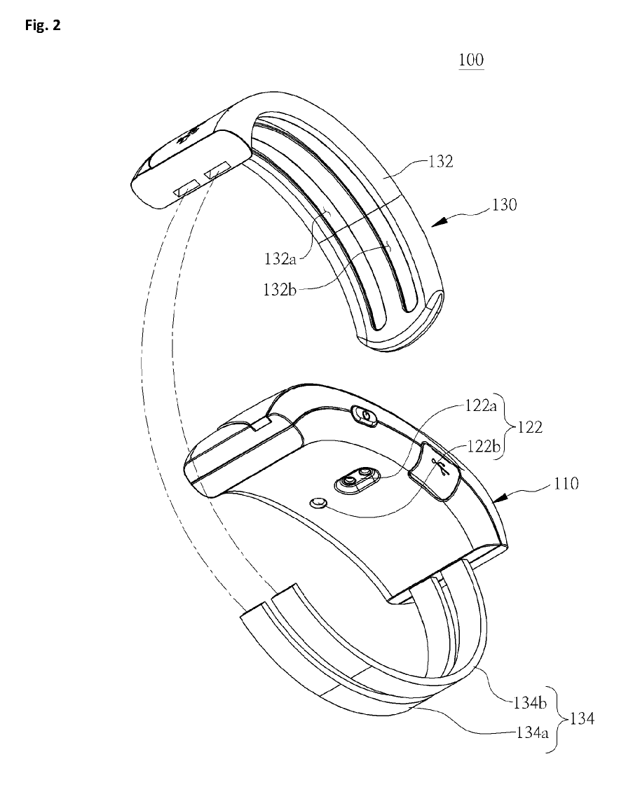

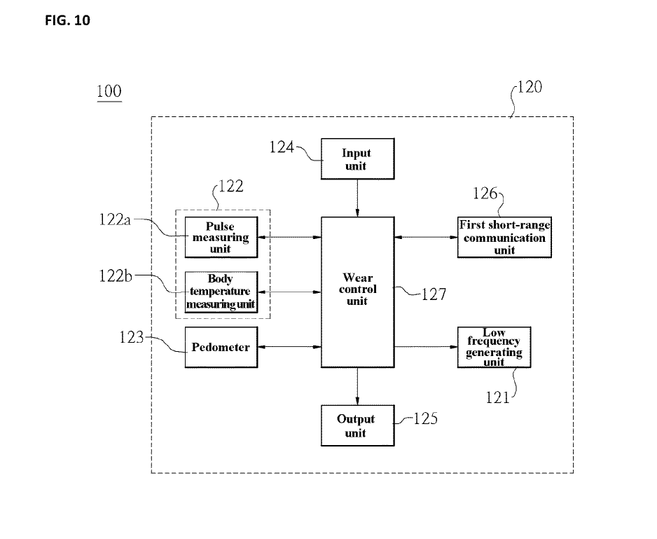

Referring to FIG. 1, a wearable device 100 having a low frequency generation function according to a preferred embodiment of the present invention is formed in a shape such as a watch worn on the wrist as a whole and includes a main body 110, a measuring unit 122 (illustrated in FIG. 2), a low frequency generating unit 121 (illustrated in FIG. 10), a belt member 130, a coupling member 140, and a treatment electrode unit 150.

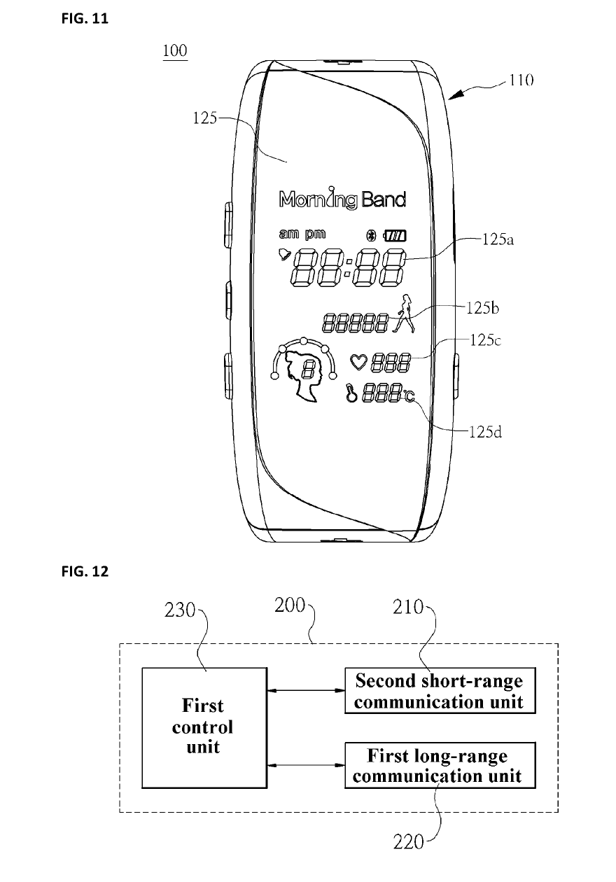

The main body 110 is positioned on the user's wrist and formed in a similar shape to a wristwatch. In addition, a measuring unit 122 is provided inside the main body 110 which is in contact with the wrist and an output unit 125 is provided outside the main body 110 which is not in contact with the wrist. The output unit 125 is configured by, for example, a liquid crystal display (LCD) for displaying an image, and will be described in detail with reference to FIG. 11 below.

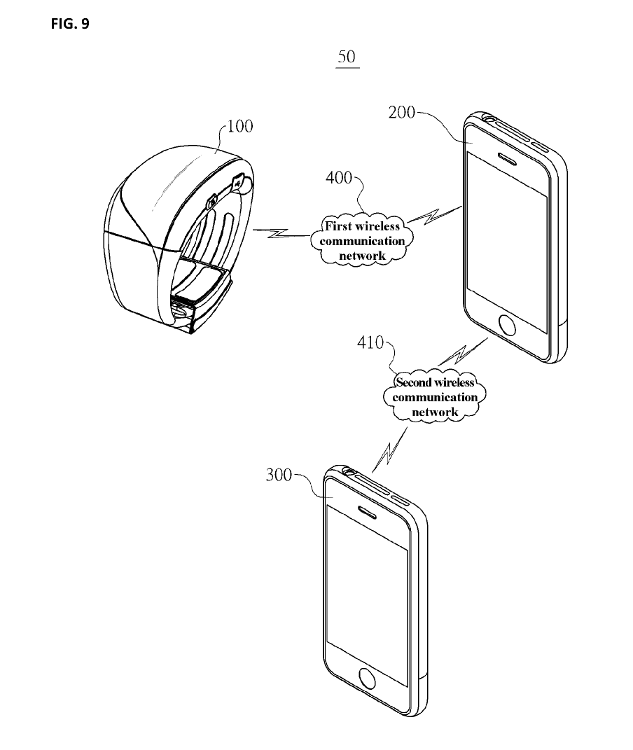

The measuring unit 122 is provided inside the main body 110 to measure biometric information of the user such as blood pressure, pulse rate, and body temperature. The measuring unit 122 includes, for example, a pulse measuring unit 122a for measuring the pulse of the user and a body temperature measuring unit 122b for measuring the body temperature of the user. The pulse measuring unit 122a may include an optical type that detects a pulse wave signal using light, a piezoelectric type that measures a pulse wave signal using pressure, and the like. In the optical type, the pulse measuring unit 122a is mounted on the inside of the main body 110 and irradiates light to the user's body to measure the user's pulse. The body temperature measuring unit 122b is configured by, for example, an infrared sensor and detects infrared rays emitted from the user and generates an electric pulse signal according to the detected infrared rays to measure the body temperature of the user. Since the low frequency generating unit 121, the pulse measuring unit 122a, and the body temperature measuring unit 122b described above are well known in the art, the more detailed description thereof will be omitted. In addition, the biometric information measured by the measuring unit 122 is transmitted to the outside to manage the health condition of the user, and this will be described below with reference to FIGS. 9 to 14.

The low frequency generating unit 121 is provided in the main body 110 and oscillates an electromagnetic pulse in a range of frequencies using an oscillation coil or the like. The low frequency generating unit 121 generates low frequency pulses of approximately 28 Hz to 33 Hz, and the generated low frequency pulses are transmitted to the treatment electrode unit 150 to be described below.

The belt member 130 is similar to a wrist strap that fixes the main body 110 to be positioned on the wrist, and may be formed of a flexible material so as to be easily bent depending on the shape of the wrist. The belt member 130 includes a first belt 132 connected to one side of the main body 110 to surround one side of the outer periphery of the wrist and a second belt 136 connected to the other side of the main body 110 to surround the other side of the outer periphery of the wrist.

The coupling member 140 couples the first belt 132 and the second belt 136 of the belt member 130 to each other and for example, couples the second belt 136 while coupling the first belt 132 to be positioned at the median nerve of the wrist.

The treatment electrode unit 150 is provided inside the coupling member 140 facing the median nerve of the wrist and includes a first electrode unit 152 and a second electrode unit 154. The first and second electrode units 152 and 154 are in contact with the median nerve of the wrist and are electrically connected to the low frequency generating unit 121 through a sliding contact portion 134 (illustrated in FIG. 2) to be described below. Accordingly, the treatment electrode unit 150 receives a low frequency generated by the low frequency generating unit 121 and transmits the received low frequency to the median nerve of the wrist again. In addition, the low frequency transmitted to the median nerve of the wrist stimulates the median nerve and then is transmitted to the central nervous system along the median nerve. Then, the central nervous system intercepts signals of nausea and vomiting and transmits stimulation to normally operate the abnormal gastrointestinal motility again.

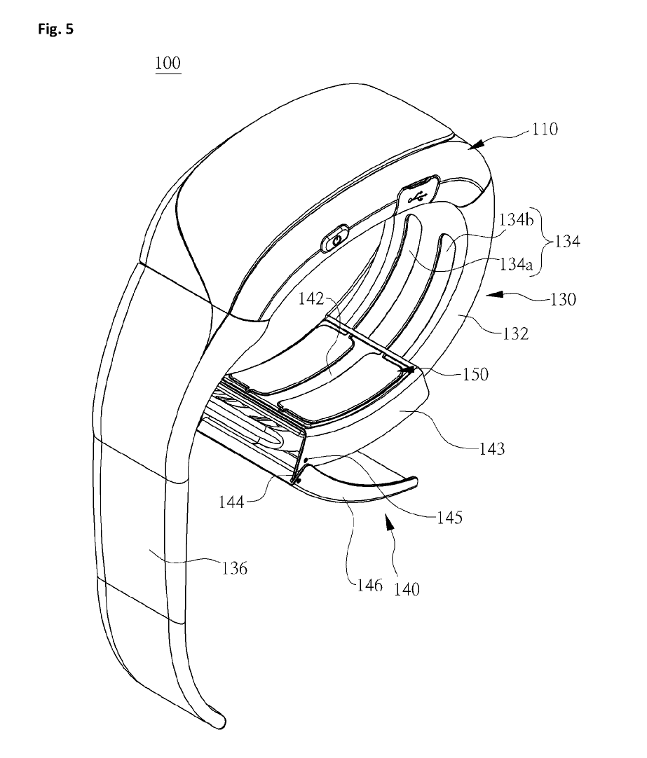

Meanwhile, since the treatment electrode unit 150 needs to be in contact with the median nerve of the wrist, the coupling member 140 to which the treatment electrode unit 150 is coupled needs also to be positioned to face the median nerve of the wrist. However, since the size of the wrist differs from person to person, the coupling member 140 needs to be positioned to be slidable along the first belt 132, and when the coupling member 140 sliding along the first belt 132 is positioned at the median nerve of the wrist, the coupling member 140 is fixed to the first belt 132 so that the coupling member 140 no longer slides. This will be described in detail with reference to FIG. 5 below.

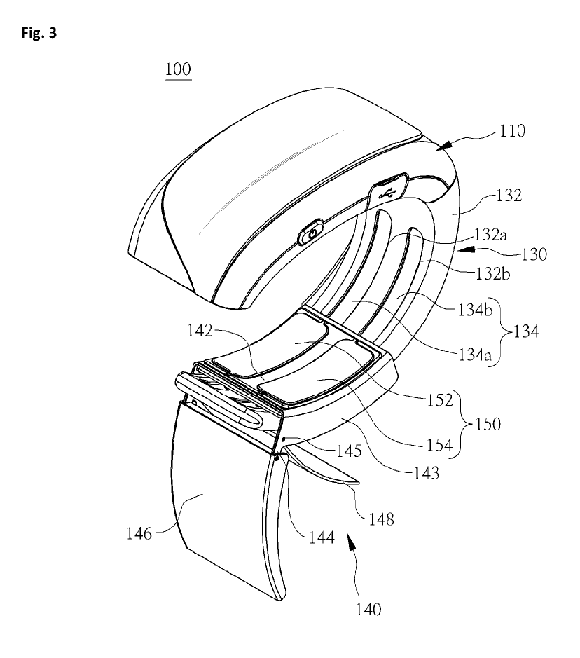

FIG. 2 is a diagram schematically illustrating a state in which a first belt is coupled to a sliding contact portion of a belt member in the wearable device having the low frequency generation function according to the preferred embodiment of the present invention and FIG. 3 is a diagram schematically illustrating a state in which a coupling guide portion of a coupling member is slidably coupled to the first belt to the belt member in the wearable device having the low frequency generation function according to the preferred embodiment of the present invention.

Referring to FIGS. 2 and 3, the belt member 130 further includes a sliding contact portion 134 in addition to the first belt 132 and the second belt 136. The sliding contact portion 134 is elongated in an inner longitudinal direction of the first belt 132 and includes a first sliding portion 134a and a second sliding portion 134b to make a pair. The first and second sliding portions 134a and 134b are made of a conductive material and one longitudinal side thereof is electrically connected to the low frequency generating unit 121 in the main body 110 and the other longitudinal side thereof is elongated in the median nerve direction of the wrist. In addition, when the first and second sliding portions 134a and 134b are inserted into the first belt 132, and when the first belt 132 is coupled to surround the first and second sliding portions 134a and 134b, first and second openings 132a and 132b are formed on the inner periphery of the first belt 132 so that the first and second sliding portions 134a and 134b are exposed to the outside.

The coupling member 140 includes a coupling guide portion 142, a coupling protrusion 144, a coupling buckle 146, a hinge coupling portion 145, and a fixing buckle 148. The coupling guide portion 142 is positioned so as to be slidable along the inner periphery of the first belt 132 and the treatment electrode unit 150 is provided inside the coupling guide portion 142 facing the wrist. In addition, when the coupling guide portion 142 slides along the inner periphery of the first belt 132, a pair of support guide portions 143 are formed to protrude along both sides of the coupling guide portion 142 so as to guide both sides of the first belt 132. In addition, the fixing buckle 148 presses the outer periphery of the first belt 132 to fix the coupling guide portion 142 sliding along the inner periphery of the first belt 132 so as to face the median nerve of the wrist, and the coupling buckle 146 presses the outer periphery of the second belt 136 so that the second belt 136 is fixed to the coupling guide portion 142.

That is, in the present invention, in addition to the coupling buckle 146 fixing the second belt 136 to the first belt 132, a fixing buckle 148 is separately provided so that the coupling guide portion 142, which slides along the inner periphery of the first belt 132, is fixed to the first belt 132. The coupling protrusion 144, the coupling buckle 146, the hinge coupling portion 145, and the fixing buckle 148 will be described in detail with reference to FIGS. 5 to 8 below.

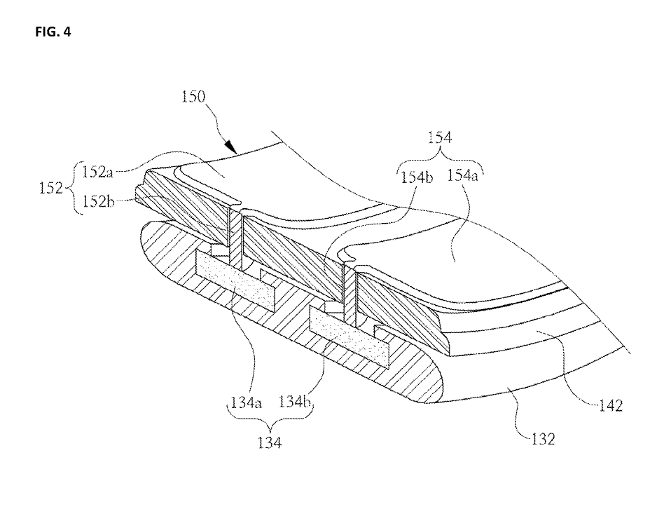

FIG. 4 is a diagram for describing a state in which the treatment electrode unit is coupled to the coupling member of the wearable device having the low frequency generation function according to the preferred embodiment of the present invention.

Referring to FIG. 4, the treatment electrode unit 150 includes a first electrode unit 152 and a second electrode unit 154. The first electrode unit 152 and the second electrode unit 154 are coupled to each other inside the coupling guide portion 142 in parallel. The first electrode unit 152 includes a first electrode 152a provided inside the coupling guide portion 142 and a first contact portion 152b electrically contacting the first sliding portion 134a of the sliding contact portion 134 by penetrating the coupling guide portion 142 at the first electrode 152a. The second electrode unit 154 includes a second electrode 154a provided in parallel with the first electrode 152a inside the coupling guide portion 142 and a second contact portion 154b electrically contacting the second sliding portion 134b of the sliding contact portion 134 by penetrating the coupling guide portion 142 at the second electrode 154a.

The first electrode 152a and the second electrode 154a are in contact with the median nerve of the wrist and formed concavely along the wrist, and may be plated with gold or the like so as to increase the conductivity and lower the resistance to the human body. The first contact portion 152b and the second contact portion 154b are configured to slide along the first sliding portion 134a and the second sliding portion 134b. When the coupling guide portion 142 slides along the inner periphery of the first belt 132 to be positioned on the median nerve of the wrist, the first contact portion 152b and the second contact portion 154b are also configured to slide along the first sliding portion 134a and the second sliding portion 134b so that the first electrode 152a and the second electrode 154a are easily positioned on the median nerve of the wrist. In addition, since the first sliding portion 134a and the second sliding portion 134b are connected to the low frequency generating unit 121 in the main body 110, the low frequency generated by the low frequency generating unit 121 is transmitted to the first electrode unit 152 and the second electrode unit 154, and the first electrode unit 152 and the second electrode unit 154 transmit the transmitted low frequency to the median nerve of the wrist again. In addition, the low frequency transmitted to the median nerve of the wrist stimulates the median nerve and then is transmitted to the central nervous system along the median nerve. Then, the central nervous system intercepts signals of nausea and vomiting and transmits stimulation to normally operate the abnormal gastrointestinal motility again.

As such, in the present invention, the low frequency generated from the low frequency generating unit 121 in the main body 110 can be easily transmitted to the median nerve of the wrist, thereby effectively preventing nausea and vomiting as side effects of anticancer therapy and chemotherapy. In addition, the present invention has an effect of effectively preventing nausea and vomiting due to motion sickness, morning sickness, or the like.

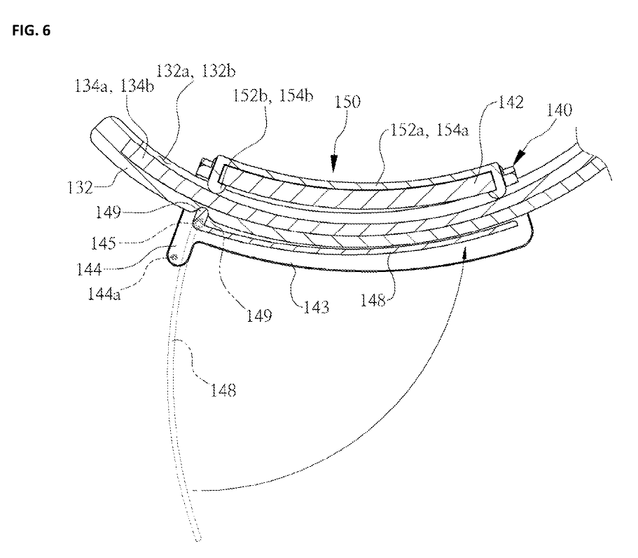

FIG. 5 is a diagram schematically illustrating a state in which the coupling member is fixed to the first belt in the wearable device having the low frequency generation function according to the preferred embodiment of the present invention and FIG. 6 is a cross-sectional view illustrating a state in which the coupling member is fixed to the first belt in the wearable device having the low frequency generation function according to the preferred embodiment of the present invention.

Referring to FIGS. 5 and 6, the coupling member 140 includes a coupling guide portion 142, a coupling protrusion 144, a coupling buckle 146, a hinge coupling portion 145, and a fixing buckle 148.

The coupling guide portion 142 is positioned to be slidable along the inner periphery of the first belt 132. In addition, a support guide portion 143 is formed to protrude along both sides of the coupling guide portion 142.

A pair of coupling protrusions 144 are formed, and when the second belt 136 is positioned to be stacked on the outer periphery of the first belt 132, the coupling protrusions 144 protrude in a direction far away from the main body 110 at both sides of the coupling guide portion 142, particularly, sides of a pair of support guide portions 143 which are positioned to be close to the end of the first belt 132 so that the leading end of the coupling protrusions 144 is provided in the outer periphery direction of the second belt 136.

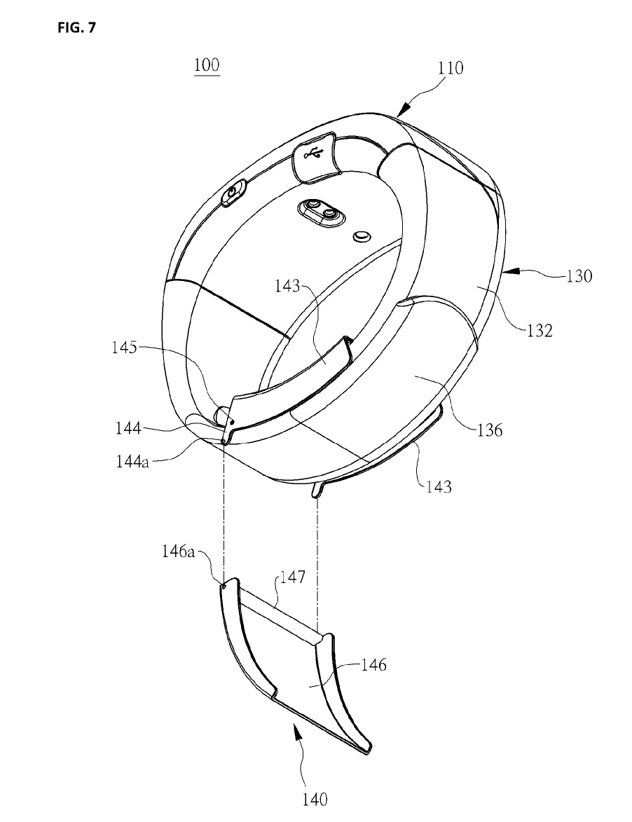

The coupling buckle 146 is hinge-coupled between the pair of coupling protrusions 144 and rotates so as to press or not to press the outer periphery of the second belt 136 positioned to be stacked on the outer periphery of the first belt 132. The coupling buckle 146 will be described with reference to FIG. 7 below.

A pair of hinge coupling portions 145 are configured and formed in a through-hole shape between each coupling guide portion 142 and each coupling protrusion 144 to face the outer periphery of the first belt 132, particularly, between each support guide portion 143 and each coupling protrusion 144.

The fixing buckle 148 is positioned to be spaced apart from the coupling guide portion 142 and the first belt 132 is inserted between the coupling guide portion 142 and the fixing buckle 148. In addition, one end of the fixing buckle 148 is hinge-coupled between the pair of hinge coupling portions 145 so that the other end of the fixing buckle 148 rotates in a direction closing to the first belt 132 or in a direction far away from the first belt 132. To this end, fixing hinge protrusions 148a are formed at both sides of one end of the fixing buckle 148 so as to be inserted into the hinge coupling portions 145 and axially coupled. In addition, when the other end of the fixing buckle 148 rotates in the direction of the first belt 132, a fixed pressing portion 149 is formed on one end of the fixing buckle 148 in a direction perpendicular to the first belt 132 and the fixed pressing portion 149 is configured to press the outer periphery of the first belt 132. As such, the fixed pressing portion 149 of the fixing buckle 148 presses the first belt 132 so that the coupling guide portion 142 is firmly fixed to the first belt 132. On the other hand, when the fixing buckle 148 rotates in the direction far away from the first belt 132, the fixed pressing portion 149 of the fixing buckle 148 is positioned in the horizontal direction to the first belt 132, the fixed pressing portion 149 does not press the first belt 132 any more, so that the coupling guide portion 142 is slidable along the first belt 132.

As such, in the present invention, since the coupling member 140 slides along the first belt 132, the coupling member 140 may be positioned to face the median nerve of the wrist, and as a result, the treatment electrode unit 150 provided in the coupling member 140 may easily transmit a low frequency to the median nerve of the wrist. Further, the coupling member 140 positioned to face the median nerve of the wrist is configured to be fixed to the first belt 132 by rotating the fixing buckle 148 to press the first belt 132, and as a result, the coupling member 140 is firmly fixed while being positioned to face the median nerve of the wrist.

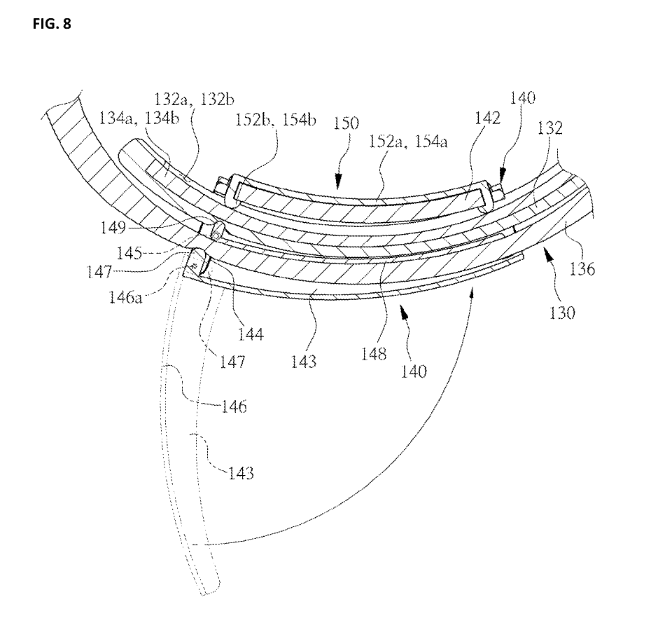

FIG. 7 is a diagram schematically illustrating a state in which a second belt is stacked on the first belt in the wearable device having the low frequency generation function according to the preferred embodiment of the present invention and FIG. 8 is a cross-sectional view illustrating a state in which a coupling buckle fixes the second belt in the wearable device having the low frequency generation function according to the preferred embodiment of the present invention.

Referring to FIGS. 7 and 8, while the fixing buckle 148 presses the first belt 132 so that the coupling guide portion 142 is fixed to the first belt 132, the second belt 136 covers the wrist, and then positioned to be staked on the first belt 132. At this time, the second belt 136 is inserted between the fixing buckle 148 and the coupling buckle 146 to be described below.

The coupling buckle 146 is positioned to be spaced apart from the fixing buckle 148 and the second belt 136 is inserted between the fixing buckle 148 and the coupling buckle 146. In addition, one end of the coupling buckle 146 is hinge-coupled between the pair of coupling protrusions 144 so that the other end of the coupling buckle 146 rotates in a direction closing to the second belt 136 or in a direction far away from the second belt 136. To this end, coupling hinge holes 146a are formed at both sides of one end of the coupling buckle 146 so as to be inserted into the hinge protrusions 144a formed on the coupling protrusion 144 and axially coupled thereto. In addition, when the other end of the coupling buckle 146 rotates in the direction of the second belt 136, a coupling pressing portion 147 is formed to protrude from one end of the coupling buckle 146 in a direction perpendicular to the second belt 136 and the coupling pressing portion 147 is configured to press the outer periphery of the second belt 136. As such, the coupling pressing portion 147 of the coupling buckle 146 presses the second belt 136 so that the second belt 136 is firmly fixed to the coupling member 140. On the other hand, when the coupling buckle 146 rotates in the direction far away from the second belt 136, since the coupling pressing portion 147 of the coupling buckle 146 is positioned in the horizontal direction to the second belt 136, the coupling pressing portion 147 does not press the second belt 136 any more, so that the second belt 136 is configured to be detached from the coupling member 140 to the outside.

As such, in the present invention, the coupling buckle 146 firmly fixes the second belt 136 inserted between the fixing buckle 148 and the coupling buckle 146, and as a result, the first belt 132 and the second belt 136 are firmly fixed to the wrist of the user while being coupled to the coupling member 140 and the main body 110 connected with the first and second belts 132 and 136 are also firmly fixed to the wrist.

Hereinafter, a health care system using a wearable device having a low frequency generation function according to a preferred embodiment of the present invention will be described with reference to the accompanying drawings.

FIG. 9 is a diagram schematically illustrating a health care system using a wearable device having a low frequency generation function according to a preferred embodiment of the present invention, FIG. 10 is a block diagram schematically illustrating the wearable device having the low frequency generation function of FIG. 9, and FIG. 11 is a diagram schematically illustrating a display unit included in the wearable device having the low frequency generation function of FIG. 9.

Referring to FIGS. 9 to 11, a health care system using a wearable device having a low frequency generation function according to a preferred embodiment of the present invention includes a wearable device 100, a first terminal 200, and a second terminal 300 so as to manage a user's health or rapidly notify a dangerous situation to a protector.

The wearable device 100 includes a managing means 120 for managing the health of the user and notifying biometric information of the user to the outside. The managing means 120 is provided in the main body 110 and includes a low frequency generating unit 121, a measuring unit 122, a pedometer 123, an input unit 124, an output unit 125, a first short-range communication unit 126 and a wear control unit 127.

The low frequency generating unit 121 generates a low frequency to transmit the low frequency to the treatment electrode unit 150. The measuring unit 122 includes a pulse measuring unit 122a and a body temperature measuring unit 122b and the pulse measuring unit 122a measures biometric information such as a pulsation or a pulse wave of the user to transmit the biometric information to the wear control unit 127. The body temperature measuring unit 122b measures a body temperature of the user to transmit the body temperature to the wear control unit 127. Since the low frequency generating unit 121 and the measuring unit 122 are described above, the detailed description thereof will be omitted.

The pedometer 123 generally checks the momentum of the user by measuring the number of steps of the user. The pedometer 123 is provided in the main body 110 and measures the momentum of the user by counting impacts generated from the outside, such as acceleration or vibration. Momentum information measured by the pedometer 123 is transmitted to the wear control unit 127.

The input unit 124 receives from the user various control signals required for controlling the low frequency generating unit 121, the measuring unit 122 or the pedometer 123, for example, a start signal, an end signal, an initialization signal, a storage signal, a setting signal, and the like, to transmit the received control signals to the wear control unit 127. In addition, the wear control unit 127 receives the control signals to control the low frequency generating unit 121, the measuring unit 122 or the pedometer 123. The input unit 124 may be formed in a keypad shape on the outside of the main body 110 or may be formed in a touch screen shape by attaching a touch panel to the output unit 125.

The output unit 125 is provided outside the main body 110 and may include an LCD, an OLED, or the like. The output unit 125 displays various characters, images, numbers, and the like under the control of the wear control unit 127. For example, the output unit 125 may include a first display unit 125a displaying time, a second display unit 125b displaying momentum information measured by the pedometer 123, a third display unit 125c displaying pulse biometric information measured by the pulse measuring portion 122a, and a fourth display unit 125d displaying body temperature biometric information measured by the body temperature measuring portion 122b. In addition, the output unit 125 may include a main speaker (not illustrated) that outputs a sound to the outside.

The first short-range communication unit 126 is configured to transmit the momentum information measured by the pedometer 123, the pulse biometric information measured by the pulse measuring unit 122a, the body temperature biometric information measured by the body temperature measuring unit 122b, to the first terminal 200 carried by the user through a first wireless communication network 400. The first wireless communication network 400 may be configured by a short-range communication network, for example, a Bluetooth, an infrared (IrDA), a wireless LAN (WLAN), or the like.

The wear control unit 127 controls the input unit 124, the low frequency generating unit 121, the measuring unit 122, the pedometer 123, the output unit 125, and the first short-range communication unit 126. That is, when a low frequency control signal for generating the low frequency is input from the input unit 124, the wear control unit 127 controls the low frequency generating unit 121 to generate the low frequency by transmitting the low frequency control signal to the low frequency generating unit 121. In addition, when a measurement control signal for measuring the pulse, the body temperature, or the momentum is input from the input unit 124, the wear control unit 127 controls the pulse measuring unit 122a, the body temperature measuring unit 122b, or the pedometer 123 to perform the measurement and outputs and displays the pulse biometric information measured by the pulse measuring unit 122a, the body temperature biometric information measured by the body temperature measuring unit 122b, or the momentum information measured by the pedometer 123, to the output unit 125. In addition, the wear control unit 127 stores a safe range for the pulse biometric information received from the pulse measuring unit 122a or the body temperature biometric information received from the body temperature measuring unit 122b, and generates a warning message when the pulse biometric information or the body temperature biometric information deviates from the safe range. In addition, the wear control unit 127 generates health information data including the pulse biometric information or the body temperature biometric information and outputs the generated health information data to the first short-range communication unit 126 so that the first short-range communication unit 126 transmits the health information data to the first terminal 200. At this time, when the wear control unit 127 generates a warning message, the wear control unit 127 includes the warning message in the health information data to control the health information data including the pulse biometric information or the body temperature biometric information and the warning message to be transmitted to the first terminal 200. In addition, the wear control unit 127 transmits the warning message to the main speaker so that the user or the persons around the user may easily determine the health condition of the user.

Meanwhile, in one embodiment of the present invention, the wear control unit 127 is configured to generate the warning message, but it is natural that the present invention is not limited thereto. In some cases, while the pulse biometric information or the body temperature biometric information is transmitted to a first control unit 230 or a second control unit 330 to be described below, the first control unit 230 or the second control unit 330 may also generate a warning message when the pulse biometric information or the body temperature biometric information deviates from the safe range.

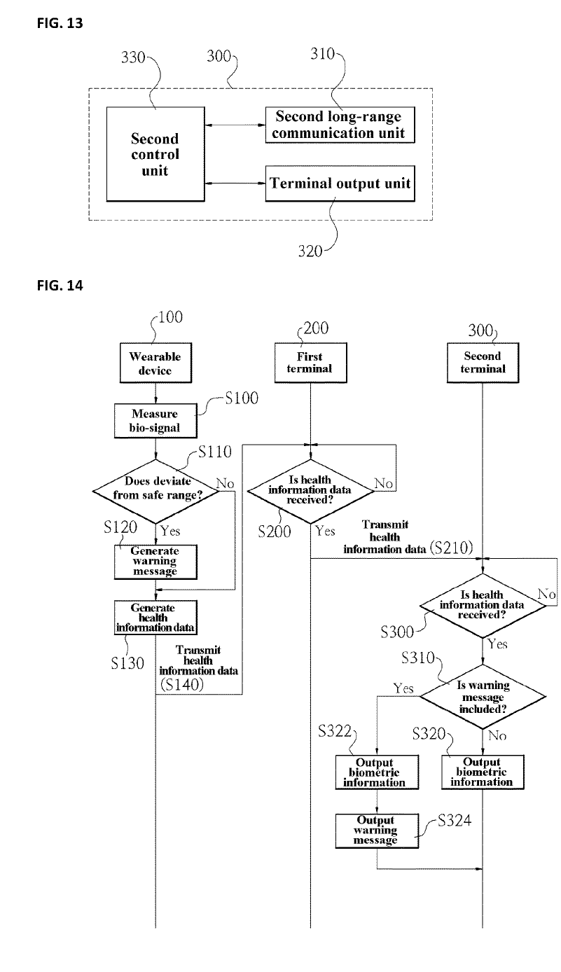

FIG. 12 is a block diagram schematically illustrating a first terminal in the health care system using the wearable device having the low frequency generation function according to the preferred embodiment of the present invention and FIG. 13 is a block diagram schematically illustrating a second terminal in the health care system using the wearable device having the low frequency generation function according to the preferred embodiment of the present invention.

Referring to FIGS. 12 and 13, the first terminal 200 includes a PDA, a mobile phone, a smart phone, and the like as a portable terminal carried by the user, and includes a second short-range communication unit 210, a first long-range communication unit 220 and a first control unit 230. The second short-range communication unit 210 is connected with the first wireless communication network 400 to wirelessly access the first short-range communication unit 126 of the wearable device 100 having the low frequency generation function. The first long-range communication unit 220 is connected with a second wireless communication network 410 to wirelessly access a second long-range communication unit 310 of the second terminal 300 to be described below. The second wireless communication network 410 may be a long-range wireless communication network, and may include at least one of a WiBro network and a mobile communication network. The mobile communication network includes at least one of a 2 generation (2G) network, a 3G network, and a 4G (long term evolution, LTE) network. The first control unit 230 is connected to the second short-range communication unit 210 so that the first short-range communication unit 126 receives the health information data including the pulse biometric information or the body temperature biometric information and the warning message transmitted to the second short-range communication unit 210. Further, the first control unit 230 controls the health information data to be transmitted to the second terminal 300 through the first long-range communication unit 220.

The second terminal 300 is configured by a PDA, a mobile phone, a smart phone, etc. like the first terminal 200, as a portable terminal managed by a protector of a user, a doctor, and the like. The second terminal 300 includes a second long-range communication unit 310, a terminal output unit 320, and a second control unit 330. The second long-range communication unit 310 is connected with the second wireless communication network 410 to wirelessly access the first long-range communication unit 220 of the first terminal 200. The terminal output unit 320 may include an LCD, a terminal speaker, a vibration unit, and the like provided in the second terminal 300. The terminal output unit 320 displays various kinds of information to the outside or outputs sound or vibration. Under the control of the second control unit 330, the terminal output unit 320 displays various characters, images, numbers and the like to the outside or outputs the sound or vibration. For example, a pulsation value of the pulse biometric information, a body temperature value of the body temperature biometric information, a warning message, and the like, which are included in the health information data, are displayed as letters or numbers, or output as sounds. The second control unit 330 is connected to the second long-range communication unit 310 so that the first long-range communication unit 220 receives the health information data including the pulse biometric information or the body temperature biometric information and the warning message transmitted to the second long-range communication unit 310 and controls the health information data to be output to the terminal output unit 320.

FIG. 14 is a schematic flowchart for describing the health care system using the wearable device having the low frequency function according to the preferred embodiment of the present invention.

Referring to FIG. 14, the measuring unit 122 of the wearable device 100 having the low frequency generation function measures biometric information such as a user's pulse or body temperature and transmits the measured biometric information to the wear control unit 127 (S100). The wear control unit 127 determines whether the biometric information transmitted from the measuring unit 122 deviates from the safe range (S110). For example, the wear control unit 127 determines whether the pulse biometric information transmitted from the pulse measuring unit 122a of the measuring unit 122, that is, a heart rate deviates from the safe range of 60 to 100 Bpm. Alternatively, the wear control unit 127 determines whether the body temperature biometric information transmitted from the body temperature measuring unit 122b of the measuring unit 122, that is, a body temperature deviates from the safe range of 30 to 40° C. In step S110, the wear control unit 127 generates a warning message when the biometric information transmitted from the measuring unit 122 deviates from the safe range (S120). The warning message may be configured by “I am a cancer patient.”, “I am a cardiac patient”, or “please help me”, so as to notify a dangerous situation of the user to the protector or neighboring persons of the user. Thereafter, the wear control unit 127 may control to output the warning message to the main speaker and notify a current dangerous situation of the user to the neighboring persons of the user. Thereafter, the wear control unit 127 generates health information data including the pulse biometric information and the body temperature biometric information of the user, and the warning messages and transmits the health information data to the first short-range communication unit 126 to control the first short-range communication unit 126 to transmit the health information data to the second short-range communication unit 210 of the first terminal 200. Meanwhile, in step S110, when the biometric information transmitted from the measuring unit 122 does not deviate from the safe range, the wear control unit 127 generates health information data including the pulse biometric information and the body temperature biometric information of the user and transmits the health information data to the first short-range communication unit 126 to control the first short-range communication unit 126 to transmit the health information data to the second short-range communication unit 210 of the first terminal 200.

Thereafter, the first control unit 230 of the first terminal 200 determines whether the health information data is received from the second short-range communication unit 210 (S200). In addition, the first control unit 230 transmits the health information data to the first long-range communication unit 220 and controls the first long-range communication unit 220 to transmit the health information data to the second long-range communication unit 310 of the second terminal 300 (S210).

Thereafter, the second control unit 330 of the second terminal 300 determines whether the health information data is received from the second long-range communication unit 310 (S300). In addition, when the health information data is received from the second long-range communication unit 310, the second control unit 330 determines whether a warning message is included in the health information data (S310). In step S310, if the warning message is included in the health information data, the second control unit 330 controls the terminal output unit 320 to output the biometric information in the health information data, and then controls the terminal output unit 320 to output the warning message in the health information data. Meanwhile, in step S310, if the warning message is not included in the health information data, the second control unit 330 controls the terminal output unit 320 to output the biometric information in the health information data.

As such, in the present invention, the wearable device 100 having the low frequency generation function is configured to measure the biometric signal of the user and notify the biometric signal to the user or the protector as well as transmit the low frequency to the median nerve of the wrist, thereby continuously monitoring the health condition of the user by the user or the protector and rapidly notifying a dangerous situation to the outside when the user is in the dangerous situation.

While the present invention has been particularly shown and described with reference to embodiments thereof, it is natural that the present invention is not limited thereto and it will be understood by those skilled in the art that various changes and modifications may be made within the technical idea of the present invention as defined by the appended claims and the technical idea belongs to the scope of the claims.

1-13. (canceled)

14. A wearable device having a low frequency generation function, the wearable device comprising:
a main body to be positioned on a wrist of a wearer and embedded with a low frequency generating unit generating a low frequency;
a wear control unit provided in the main body to control the low frequency generating unit to generate the low frequency;
a belt member having a first belt connected to one side of the main body to surround one side of the outer periphery of the wrist and a second belt connected to the other side of the main body to surround the other side of the outer periphery of the wrist;
a coupling member coupling the first belt and the second belt; and
a treatment electrode unit provided to a portion of the coupling member facing the wrist and transmitting the low frequency generated by the low frequency generating unit to the wrist.

15. The wearable device according to claim 14, wherein the coupling member comprises:
a coupling guide portion coupled to the first belt and having the treatment electrode unit at the portion facing the wrist;
a pair of coupling protrusions protruding from both sides of the coupling guide portion in a direction of the outer periphery of the second belt which is positioned to be stacked on the outer periphery of the first belt; and
a coupling buckle hinge-coupled between the pair of coupling protrusions and rotating to press or not the outer periphery of the second belt which is positioned to be stacked on the outer periphery of the first belt.

16. The wearable device according to claim 15, wherein the belt member further includes:
a sliding contact portion provided in an inner longitudinal direction of the first belt, positioned to be exposed to the outside along the inner periphery of the first belt, and electrically connected to the low frequency generating unit, and
wherein the coupling member further includes the coupling guide portion coupled to be slidable along the inner periphery of the first belt; a pair of hinge coupling portions provided between the coupling guide portion and the coupling protrusion to face the outer periphery of the first belt; and a fixing buckle hinge-coupled between the pair of hinge coupling portions and rotating to press or not the outer periphery of the first belt, and
the treatment electrode unit includes an electrode portion positioned at the coupling guide portion and a contact portion protruding from the electrode portion in the sliding contact portion direction by penetrating the coupling guide portion.

17. The wearable device according to claim 16, wherein a pair of treatment electrode units are configured and a pair of sliding contact portions are configured to contact with the treatment electrode units, respectively.

18. The wearable device according to claim 14, wherein the treatment electrode unit is positioned to face the median nerve of the wrist.

19. The wearable device according to claim 14, wherein a measuring unit measuring biometric information of the user is provided inside the main body facing the wrist.

20. The wearable device according to claim 19, wherein an output unit outputting various data to the outside is further included outside the main body positioned at an opposite side to the wrist, and the wear control unit outputs the biometric information measured by the measuring unit to the output unit.

21. The wearable device according to claim 19, wherein the measuring unit measures a pulse at the wrist of the wearer or a body temperature at the wrist of the wearer.

22. The wearable device according to claim 14, wherein a pedometer is provided in the main body.

23. A health care system comprising the wearable device having a low frequency generation function according to claim 19;
wherein the wear control unit receives biometric information from the measuring unit, and comprising a first short-range communication unit receiving health information and biometric data from the wear control unit to output the received health information data to the outside through a first wireless communication network;
a first terminal including a second short-range communication unit connected wirelessly to the first wireless communication network and receiving the health information data from the first short-range communication unit, a first long-range communication unit outputting the health information data to the outside through a second wireless communication network, and a first control unit controlling the first long-range communication unit to output the health information data received from the second short-range communication unit to the outside; and
a second terminal including a second long-range communication unit connected wirelessly to the second wireless communication network and receiving the health information data from the first long-range communication unit, a terminal output unit outputting the health information data received from the second long-range communication unit to the outside, and a second control unit controlling the terminal output unit to output the health information data received from the second long-range communication unit to the outside.

24. The health care system according to claim 23, wherein the first wireless communication network is configured by a wireless communication network using Bluetooth and the second wireless communication network is configured by a mobile communication network including any one selected from a 2G network, a 3G network, and a 4G network (an LTE network).

25. The health care system according to claim 23, wherein any one control unit selected from the wear control unit, the first control unit, and the second control unit stores a safe range for the biometric information and generates a warning message when the biometric information deviates from the safe range, and the warning message is included in the health information data.

26. The health care system according to claim 25, wherein the terminal output unit includes a speaker outputting the warning message as a sound or a vibration unit outputting the warning message as a vibration, or a display unit outputting the warning message as an image.
